(19) United States
(12) Patent Application Publication
(10) Pub. No.: US 2021/0045751 A1
(43) Pub. Date: Feb. 18, 2021

(54) DEVICE, METHOD, AND KIT FOR PERIHEPATIC PACKING

(71) Applicant: UNITY HEALTH TORONTO, TORONTO (CA)

(72) Inventor: João DE REZENDE NETO, TORONTO (CA)

(73) Assignee: UNITY HEALTH TORONTO, TORONTO, ON (CA)

(21) Appl. No.: 16/979,953

(22) PCT Filed: Mar. 21, 2019
(86) PCT No.: PCT/CA2019/050345

§ 371 Date: Sep. 11, 2020

Related U.S. Application Data
(60) Provisional application No. 62/646,941, filed on Mar. 23, 2018.

(51) Int. Cl. A61B 17/135

(52) U.S. Cl. CPC A61B 17/135; A61B 2017/00561

(57) ABSTRACT

A kit of parts for perihepatic packing includes a sac, at least one securing device, and a first inflation/deflation device. The sac has front and back walls, and a periphery. The front and back walls are joined together along the periphery to define an enclosure. The periphery has top and bottom edges, and the sac is foldable to a folded configuration in which the top and bottom edges are brought towards each other. The top edge has a notch extending inwardly towards the bottom edge. The sac has first and second side portions on first and second sides of the notch. At least a portion of the sac is translucent. The securing device facilitates securing of the sac in the folded configuration. The first inflation/deflation device adds a fluid to the enclosure to inflate the sac or removes the fluid from the enclosure to deflate the sac.

### CROSS REFERENCES TO RELATED APPLICATIONS

This application claims the benefit of U.S. Provisional Patent Application No. 62/646,941 filed on Mar. 23, 2018, the entire contents of which are incorporated herein by reference.

### FIELD

This document relates to treatment of hepatic bleeding. More specifically, this document relates to devices, methods, and kits for perihepatic packing.

### BACKGROUND

US Patent Application Publication No. 2006/0015004 (Sitzmann) discloses methods and devices for hemostatic control of an injured internal organ. In one embodiment, a container is provided for at least partially surrounding an injured organ and exerting a compressive force upon the organ. Methods of treatment utilizing such devices are also provided.

U.S. Pat. No. 5,057,117 (Atweh) discloses methods and apparatus for compartmentalizing and carrying out hemostasis of a massively bleeding internal bodily organ of a patient. Generally, the apparatus includes a flexible compartment-defining structure having physical dimensions sufficient to generally conform to the gross geometry of a substantial portion of the organ. The flexible compartment-defining structure has an inner and outer surface, and is configurable so as surround and compartmentalize a substantial portion of the organ, with the inner surface facing the external surface of the organ. The apparatus can be used to carry out hemostasis and/or the collection of blood for reprocessing and subsequent introduction to the patient by auto-transfusion. In another embodiment, the apparatus manipulates the surface-temperature of an internal bodily organ. Such apparatus further includes a flexible fluid-containable structure disposed over a substantial portion of the outer surface of the flexible compartment-defining structure, and is adapted for the passage of a gas or fluid therethrough in order to control the surface-temperature of the organ.

### SUMMARY

The following summary is intended to introduce various aspects of the detailed description, but not to define or delimit any invention.

According to some aspects, a kit of parts for perihepatic packing includes a sac having a front wall, a back wall, and a periphery. The front wall and back wall are joined together along the periphery to define an enclosure between the front wall and the back wall. The periphery has a top edge and a bottom edge, and the sac is foldable to bring the top edge and the bottom edge towards each other. The top edge has a notch extending inwardly towards the bottom edge and the sac has a first side portion on a first side of the notch and a second side portion on a second side of the notch. At least a portion of the sac is translucent. The kit further includes at least one securing device for facilitating securing of the top edge and the bottom edge together. The kit further includes a first inflation device for adding a fluid to the enclosure to inflate the sac.

In some examples, the entirety of the sac is translucent.

In some examples, the sac has a sac height between the top edge and the bottom edge, and the notch has a notch height. The notch height can be between one third and two thirds of the sac height.

In some examples, the first side portion is larger than the second side portion.

In some examples, the at least one securing device includes a first set of securing devices along the top edge of the sac and a second set of securing devices along the bottom edge of the sac. The at least one securing device can further include at least one auxiliary device for securing the first set of securing devices to the second set of securing devices. The sac can include a first set of eyelets forming the first set of securing devices and a second set of eyelets forming the second set of securing devices. The sac can include a first set of grommets forming the first set of eyelets, and a second set of grommets forming the second set of eyelets. The auxiliary device can include at least one tie for releasably tying the first set of securing devices to the second set of securing devices. The ties can be laceable between the first set of eyelets and the second set of eyelets to tie the top edge to the bottom edge. The ties can include umbilical tape and/or surgical vessel loops. The kit can further include at least one lock for securing the ties.

In some examples, the kit further includes a pressure gauge for measuring the pressure in the enclosure.

In some examples, the inflation device includes a manual air pump.

In some examples, the first inflation device is in communication with the enclosure on the first side portion, and the kit of parts further includes a second inflation device in communication with the enclosure on the second side portion.

In some examples, the sac is a polyurethane sac.

According to some aspects, a device for perihepatic packing includes a sac having a front wall, a back wall, and a periphery. The front wall and back wall are joined together along the periphery to define an enclosure between the front wall and the back wall. The periphery has a top edge and a bottom edge, and the sac is foldable to bring the top edge and the bottom edge towards each other. The top edge includes a notch extending inwardly towards the bottom edge and the sac has a first side portion on a first side of the notch and a second side portion on a second side of the notch. At least a portion of the sac is translucent. The device includes least one inflation port in the sac for adding a fluid to the enclosure. A first set of securing devices is provided along the top edge and a second set of securing devices is provided along the bottom edge, for facilitating securing of the top edge and the bottom edge together.

In some examples, an entirety of the sac is translucent.

In some examples, the sac has a sac height between the top edge and the bottom edge, and the notch has a notch height, and the notch height is between one third and two thirds of the sac height.

In some examples, the first side portion is larger than the second side portion.

In some examples, the device includes a first set of eyelets forming the first set of securing devices and a second set of eyelets forming the second set of securing devices. A first set of grommets can form the first set of eyelets, and a second set of grommets can form the second set of eyelets.

In some examples, the sac is a polyurethane sac.

In some examples, the at least one inflation port includes a first inflation port on the first side portion, and a second inflation port on the second side portion.

According to some aspects, a method for perihepatic packing includes: a) positioning a sac proximate a bleeding liver. The sac has a first side portion and a second side portion, a first edge extending along the first side portion and second side portion and defining a notch between the first side portion and second side portion, and a second edge extending along the first side portion and second side portion opposite the first edge. The method further includes b) tucking a section of the first side portion behind a right lobe of the liver, c) tucking a section of the second side portion behind the left lobe of the liver, d) positioning the notch to receive the hepatic veins, e) bringing the section of the first side portion and the section of the second side portion around the liver towards the second edge, f) securing the first edge to the second edge, and g) inflating the sac.

In some examples, at least a portion of the sac is translucent, and the method further includes viewing the liver through the sac to monitor the status of the liver and/or detect any bile leakage. The method can further include adjusting an inflation level in the sac in response to the status of the liver to control bleeding.

In some examples, step b) includes tucking the section of the first side portion over the top of the liver and step c. includes tucking the section of the second side portion over the top of the liver.

In some examples, step f) includes lacing at least one tie between the first edge and the second edge.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a top plan view of a kit of parts for perihepatic packing;

FIG. 2 is a bottom plan view of the kit of parts of FIG. 1;

FIG. 3 is a cross-section showing the device and ties of FIG. 1 in use, wrapped around a liver and inflated;

FIG. 4 is a perspective view showing the kit of parts of FIG. 1 in use, wrapped around a liver and inflated; and

FIG. 5 is a top plan view of an alternative device for perihepatic packing.

### DETAILED DESCRIPTION

Various apparatuses or processes will be described below to provide an example of an embodiment of the claimed subject matter. No embodiment described below limits any claim and any claim may cover processes or apparatuses that differ from those described below. The claims are not limited to apparatuses or processes having all of the features of any one apparatus or process described below or to features common to multiple or all of the apparatuses described below. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Disclosed herein are devices for perihepatic packing, as well as related kits and methods. The device can be used to stop or reduce or minimize hepatic bleeding, which might occur after trauma to the liver. In general, the device can be wrapped around the liver (either partially or fully), and can be inflated to apply pressure to the liver, to treat bleeding. Alternatively, the device can be wrapped around the liver (either partially or fully), and fluid (e.g. air) can be vacuumed from the device, to apply pressure to the liver to treat bleeding. The level of inflation or vacuum can be adjusted to optimize the pressure applied to the liver.

The device or a portion thereof can be translucent, so that the liver can be viewed during treatment. This can allow ischemia of the liver to be detected while the device is in place, and/or for continued bleeding to be detected while the device is in place, and/or for bile leakage to be detected while the device is in place. If ischemia is detected and it is believed that the ischemia is being caused by excessive pressure applied by the device, the pressure applied by the device can be reduced. If continued bleeding is detected and it is believed that the continued bleeding is being caused by insufficient pressure applied by the device, the pressure applied by the device can be increased. If bile leakage is detected, this can potentially indicate injury to larger biliary channel that might require repair at the time of removal of the device.

The device can have a generally smooth surface, and can be made from a plastic that does not adhere to the surface of the liver, preventing dislodgement of the clot and further damage to the liver upon removal of the device. For example, the device can be made from polyurethane. In some examples, the device can be coated with a pro-coagulant, to encourage clotting.

The device can be shaped so that both lobes of the liver can be pressurized, without substantially pressurizing the retrohepatic vena cava and/or the hepatic veins.

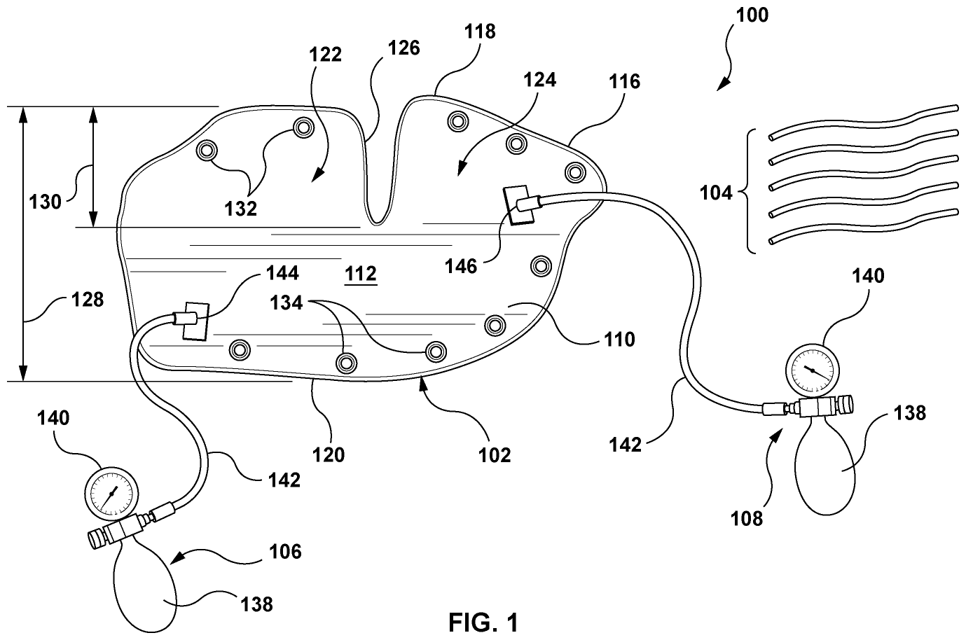

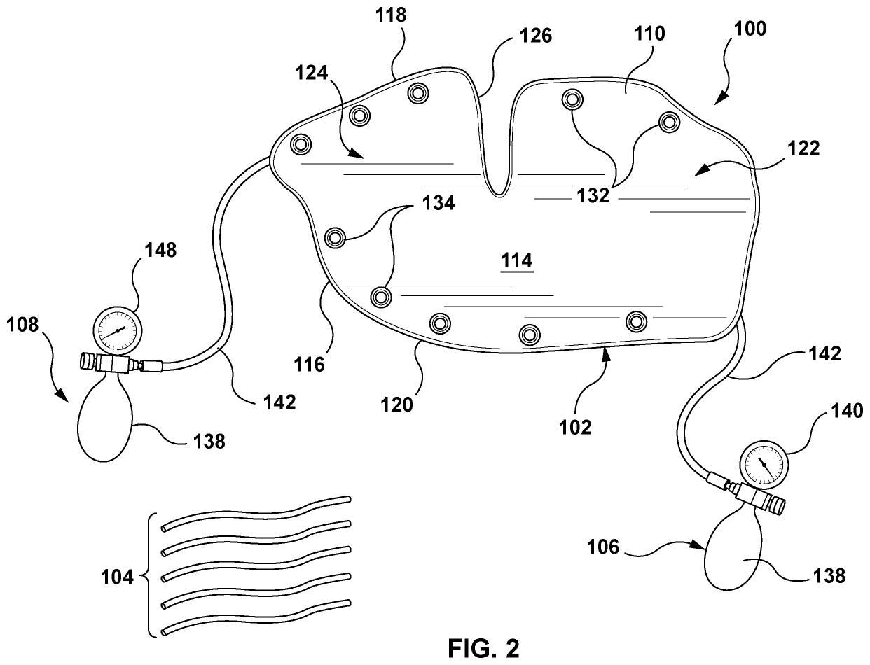

Referring now to FIGS. 1 and 2, an example kit of parts 100 for perihepatic packing is shown. The kit 100 includes a device 102 for applying pressure to the liver, and accessories in the form of ties 104, and first 106 and second 108 inflation devices.

The device 102 and the accessories can be sold together as a kit, or can be sold separately. In the examples shown, the accessories are formed separately from the device 102 and are removable from the device 102. In alternative examples, one or more of the accessories can be integral with the device 102 or permanently secured to the device 102.

Referring still to FIGS. 1 and 2, in the example shown, the device 102 includes a sac 110, which is translucent. The sac 110 may be made from plastic. For example, the sac 110 may be made from translucent polyurethane. The polyurethane can have a thickness of, for example, between 120 microns and 200 microns (inclusive). As used herein, the term ‘translucent’ indicates that the sac 110 is sufficiently see-through to allow a user to view a liver through the sac 110 and determine the status of the liver (e.g. whether it is necrotic or whether it is bleeding). The term ‘translucent’ includes examples in which the sac is completely transparent, as well as examples in which the sac is only partially see-through (e.g. wherein the sac has a slightly frosted appearance).

In the example shown, the entirety of the sac 110 is translucent. In alternative examples, only a portion of the sac may be translucent. For example, the sac may have one or more translucent windows.

Referring still to FIGS. 1 and 2, in the example shown, the sac 110 has a front wall 112 (shown in FIG. 1), a back wall 114 (shown in FIG. 2), and a periphery 116. The front wall 112 and back wall 114 are joined together along the periphery 116 to define an enclosure between the front 112 and the back wall 114. The sac 110 can be inflated by adding a fluid (e.g. air) to the enclosure. The front wall 112 and back wall 114 may, for example, be formed from separate panels of plastic that are joined together along a seam (e.g. by adhering or welding or stitching), or may be made from a single integral panel of plastic. Alternatively, the front wall and back wall may be formed from separate panels of plastic that are joined together by an intermediate panel of plastic (e.g. by adhering or welding or stitching the three panels together).

When uninflated, the sac 110 may be generally flat, so that the front wall 112 and back wall 114 lie against each other. When inflated, the front wall 112 and back wall 114 may be spaced apart, for example by up to 0.5 cm or up to 1 cm or up to 2 cm or more.

Referring still to FIGS. 1 and 2, the periphery 116 has a first edge 118 and a second edge 120, which are also referred to herein as a “top edge 118” and a “bottom edge 120”, respectively. The terms “top” and “bottom” with respect to the edges are used herein for convenience with regard to the drawings. It is not necessary that the device always be used with the top edge above the bottom edge, and if the device is inverted, the first edge 116 may still be referred to as a “top edge 116”.

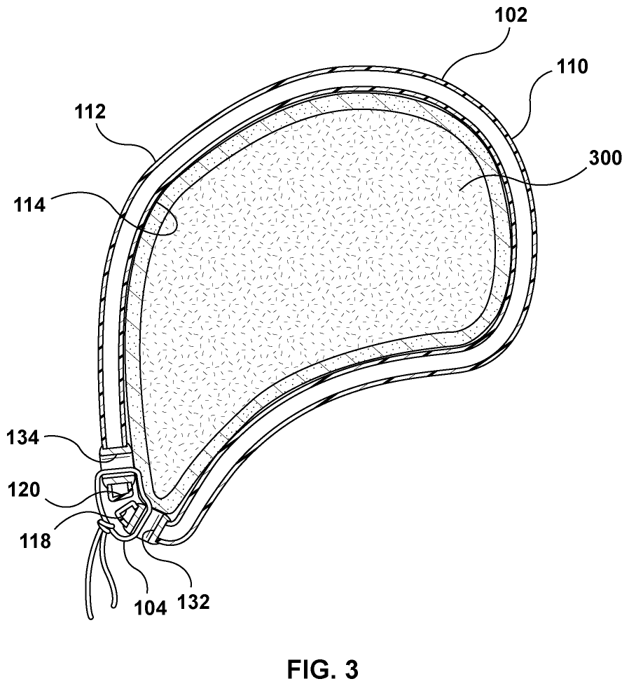

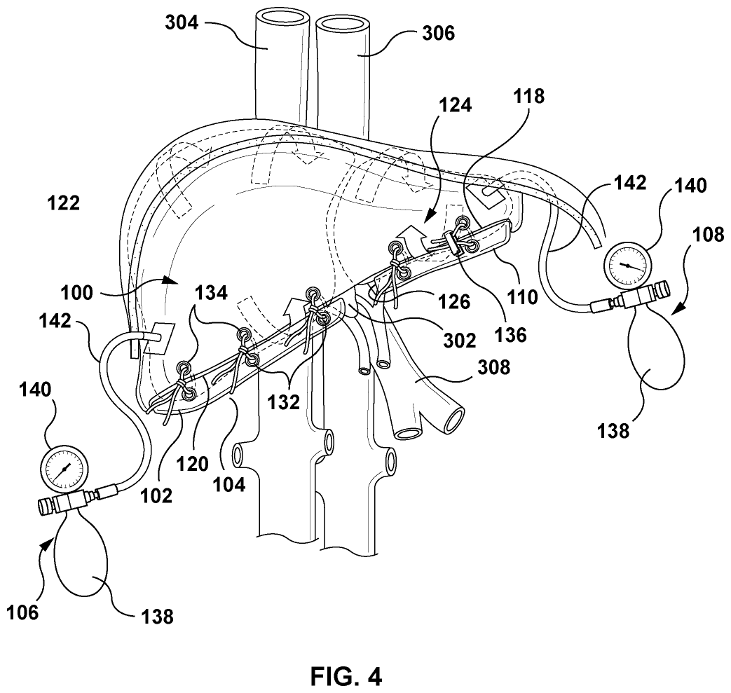

In the example shown, the sac 110 is flexible, so that it can be folded to bring the top edge 118 and bottom edge 120 towards each other, as shown in FIGS. 3 and 4 (which will be described in further detail below). That is, the sac 110 can be folded around a liver and can generally conform to the shape of the liver, to bring the top edge 118 and bottom edge 120 towards each other. This configuration—i.e. when the top edge 118 and bottom edge 120 are brought towards each other—can be referred to as a “folded configuration”.

Referring still to FIGS. 1 and 2, the sac 110 has a first side portion 122 and a second side portion 124. The top edge 118 includes a notch 126, which extends inwardly towards the bottom edge 120, and the first side portion 122 and second side portion 124 are on opposite sides of the notch 126. In the example shown, the notch 126 is generally U-shaped. In other examples, the notch may be another shape, such as V-shaped or rectangular or any other shape that defines a space or gap between the first side portion and second side portion. The notch 126 is sized and positioned so that when the first side portion 122 is wrapped around the right lobe of a liver and the second side portion 124 is wrapped around the left lobe of a liver, the notch 126 accommodates the hepatic veins, with the retrohepatic vena cava and hepatic veins in the notch, and with the first side portion 122 and second side portion 124 spaced from the porta hepatis. By resting in the notch 126, when the sac 110 is inflated, the retrohepatic vena cava and hepatic veins are not substantially compressed.

In some examples, the sac 110 has a height 128 (also referred to as a “sac height”) between the top edge 118 and the bottom edge 120, and the notch 126 has a height 130 (also referred to as a “notch height”) that is between about one-third and two-thirds of the height 128 of the sac 110. In the example shown, the notch height 130 is about one half of the height 128 of the sac 110.

Referring still to FIGS. 1 and 2, in the example shown, the first side portion 122 is larger than the second side portion 124, since the right lobe of the liver is generally larger than the left lobe of the liver.

In general, at least one securing device may be provided to facilitate securing of the sac 110 in the folded configuration when the sac 110 is wrapped around the liver. In some examples, the securing device may secure the top edge 118 to the bottom edge 120 when the sac 110 is wrapped around the liver. For example, the device can include a first set of securing devices along the top edge of the sac, and a second set of securing devices along the bottom edge of the sac. The first set of securing devices and second set of securing devices may secure directly to each other (e.g. the first set of securing devices may include a first set of clips, and the second set of securing devices may include a second set of clips, and the clips of the first set may mate with the clips of the second set). Alternatively, one or more auxiliary devices (e.g. ties such as ties 104) may secure the first set of securing devices to the second set of securing devices. In the example shown, the device 102 includes a first set of securing devices 132 (only some of which are labelled in the Figures) along the top edge 118, and a second set of securing devices 134 (only some of which are labelled) along the bottom edge 120. The securing devices 132 and 134 are in the form of eyelets. The ties 104 can be laced between the eyelets along the top edge 118 and the eyelets along the bottom edge 120 and knotted, to tie the top edge 118 to the bottom edge 120 (as is shown in FIGS. 3 and 4).

The ties 104 can be any suitable material that can releasably tie the top edge 118 to the bottom edge 120. For example, the ties 104 can be strips of medical tape such as umbilical tape, surgical vessel loops, or pieces of a suture material.

In the example shown, the ties 104 can be knotted together to tie the top edge 118 to the bottom edge 120. In other examples, ties can be secured together in another manner. For example, a lock (e.g. the cord lock toggle 136 shown in FIG. 4) may be used to tighten the ties 104 and secure them in place. Alternatively, the ends of the ties 104 may clip or snap together.

In the example shown, the eyelets are formed by grommets that are mounted to the sac 110. In alternative examples, the eyelets can be of another configuration.

Referring still to FIGS. 1 and 2, as mentioned above, the kit further includes first 106 and second 108 inflation devices for adding a fluid (e.g. air) to the enclosure to inflate the sac 110. In the example shown, the first 106 and second 108 inflation devices each include a respective manual air pump in the form of a bulb 138 that is manually compressed to inflate the sac 110, and a respective pressure gauge 140 for measuring the pressure in the enclosure. Each bulb 138 is connected to the enclosure via a respective tube 142. Valves may be used to maintain the system closed to prevent air leak through the tube and backflow into the bulb. In the example shown, the tube 142 of the first inflation device 106 is connected to the enclosure on the first side portion 122 of the sac 110, via an inflation port 144, and the tube 142 of the second inflation device 108 is connected to the enclosure on the second side portion 124 of the sac 110, via an inflation port 146.

In alternative examples, the inflation devices may be of another configuration. For example, the inflation devices may include one or more automatic pumps, and/or one or more digital pressure gauges, and/or may be configured to add an alternative fluid to the sac (e.g. saline). In further alternative examples, only a single inflation device may be provided.

In further alternative examples, the kit can include a vacuum device instead of an inflation device. The vacuum device can vacuum fluid from the enclosure, to cause the sac to shrink and tighten around the liver. In such examples, the ports of the sac may be referred to as vacuum ports instead of inflation ports.

In general, in this document, the term “inflation/vacuum device” is used to refer to a device that can inflate the enclosure, or that can vacuum the enclosure, or that can do both.

An example method for perihepatic packing will now be described. The method will be described with reference to the kit 100 of FIGS. 1 and 2; however, the method is not limited to use with the kit 100, and the kit 100 is not limited to use according to the example method. The method will be described with reference to FIGS. 3 and 4, in which FIG. 3 shows the liver 300 in general, and FIG. 4 shows a falciform ligament 302, the inferior vena cava 304, the aorta 306, and the hepatic portal veins 308. In FIG. 4, the right and left lobes of the liver 300 can be seen through the sac 110.

In general, the kit 100 and method may be used to treat bleeding of the liver, such as may occur after trauma (e.g. automobile accidents, stabbings, gunshot wounds, etc.) or after elective surgical procedures on the liver, and also from hepatic bleeding unrelated to surgical or invasive procedures. A patient experiencing such bleeding may be laparotomized, and the sac 110 may be placed proximate the bleeding liver, so that it generally overlies the liver with the back wall 114 facing towards the liver and the bottom edge 120 adjacent the caudal end of the liver.

As shown in FIGS. 3 and 4, the top section of the first side portion 122 may then be tucked posteriorly, behind the right lobe of the liver, and the top section of the second side portion 124 may be tucked posteriorly, behind the left lobe of the liver. The notch 126 may receive the falciform ligament 302 of the liver and the hepatic veins, and may be positioned so that upon inflation of the device, compression of the retrohepatic vena cava is prevented. The first side portion 122 and second side portion 124 may remain spaced from the porta hepatis and from the hepatic portal veins 308 and from the retrohepatic inferior vena cava 304.

The top section of the first side portion 122 and the top section of the second side portion 124 may then be brought around the caudal end of the liver 300, towards the bottom edge 120 of the sac.

Referring still to FIGS. 3 and 4, with the sac 110 wrapped around the liver 300, the top edge 120 may be secured to the bottom edge 124, for example by lacing ties 104 between the top edge 120 and the bottom edge 124 through the grommets, and tying or otherwise locking the ties 104.

With the sac 110 secured in place, the inflation devices 106, 108 may be used to inflate the sac 110 and apply pressure to the liver. Since the sac 110 is translucent, the liver 300 may be viewed through the sac 110, and the status of the liver 300 may be monitored during inflation. The inflation level of the sac 110 may be adjusted in response to the status of the liver. For example, if continued bleeding is observed, inflation may be continued, in order to apply greater pressure to the liver. Alternatively, if the stoppage of bleeding is viewed, inflation may be stopped. Alternatively, if liver ischemia is viewed, the level of inflation may be decreased.

The device 102 may remain on the liver 300 for an extended period of time, for example several days, until the liver 300 is stabilized. During this time, with the patient's abdomen remaining open, the liver 300 may be periodically viewed through the sac 110, and the level of inflation may be adjusted.

In addition to or as an alternative to adjusting the amount of pressure applied to the liver by adjusting the level of inflation, the amount of pressure applied to the liver can be adjusted by tightening or loosening the ties.

In alternative examples, the device 100 may be used in a similar fashion as described above, but inverted (i.e. the device may be wrapped around the liver from the bottom).

The device 100 may also have other uses, such as in tamponade pressure from pelvic fractures.

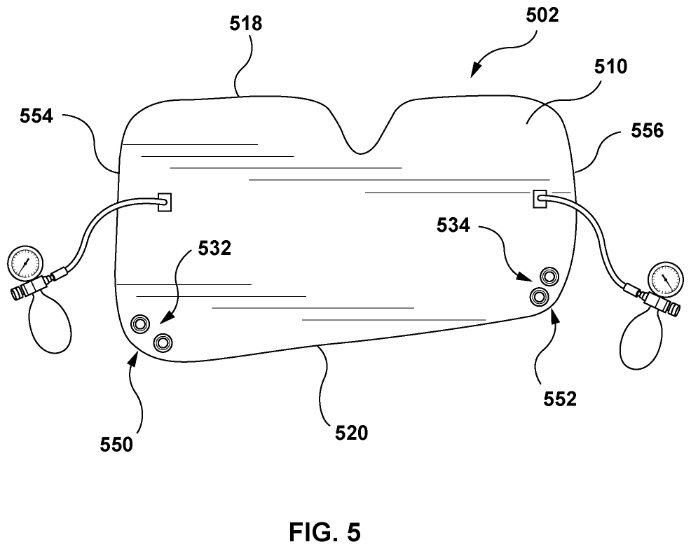

Referring now to FIG. 5, an alternative device 502 for applying pressure to the liver is shown. In FIG. 5, features that are like those of FIGS. 1 to 4 are referred to with like reference numerals, incremented by 400.

In the device 500, the sac 510 is slightly larger than the sac 110 of FIGS. 1 to 4, and the overall shape of the sac 510 is generally rectangular. Furthermore, the securing devices include a first set of securing devices 532, in the form of eyelets, proximate the first bottom corner 550 of the sac 510, and a second set of securing devices 534, in the form of eyelets, proximate the second bottom corner 552 of the sac 510.

The device 500 can be used in a similar fashion to the device 100. However, in addition to tucking the top edge 518 of the sac posteriorly, the bottom edge 520 and side edges 554 and 556 of the sac can also be tucked posteriorly, and then the sac 510 can be secured in the folded position by lacing ties between the first set of securing devices 532 and second set of securing devices 534.

As described above, the sac can then be inflated to apply pressure to the liver

While the above description provides examples of one or more processes or apparatuses, it will be appreciated that other processes or apparatuses may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

1. A kit of parts for perihepatic packing, comprising:
a sac having a front wall, a back wall, and a periphery, wherein the front wall and back wall are joined together along the periphery to define an enclosure between the front wall and the back wall, wherein the periphery has a top edge and a bottom edge and the sac is foldable to a folded configuration in which the top edge and the bottom edge are brought towards each other, wherein the top edge comprises a notch extending inwardly towards the bottom edge and the sac has a first side portion on a first side of the notch and a second side portion on a second side of the notch, and wherein at least a portion of the sac is translucent;
at least one securing device for facilitating securing of the sac in the folded configuration; and
a first inflation/vacuum device for adding a fluid to the enclosure to inflate the sac and/or for vacuuming a fluid from the enclosure to vacuum the sac.

2. The kit of parts of claim 1, wherein the entirety of the sac is translucent.

3. The kit of parts of claim 1, wherein the sac has a sac height between the top edge and the bottom edge, and the notch has a notch height, and the notch height is between one third and two thirds of the sac height.

4. The kit of parts of claim 1, wherein the first side portion is larger than the second side portion.

5. The kit of parts of claim 1, wherein the at least one securing device comprises a first set of securing devices along the top edge of the sac and a second set of securing devices along the bottom edge of the sac, or a first set of securing devices on the first side portion of the sac and a second set of securing devices on the second side portion of the sac.

6. (canceled)

7. The kit of parts of claim 5, wherein the at least one securing device further comprises at least one auxiliary device for securing the first set of securing devices to the second set of securing devices.

8. The kit of parts of claim 7, wherein the auxiliary device comprises at least one tie for releasably tying the first set of securing devices to the second set of securing devices.

9. (canceled)

10. (canceled)

11. The kit of parts of claim 8, wherein the sac comprises a first set of eyelets forming the first set of securing devices and a second set of eyelets forming the second set of securing devices, and the ties are laceable between the first set of eyelets and the second set of eyelets to tie the first set of securing devices to the second set of securing devices and secure the sac in the folded configuration.

12. (canceled)

13. The kit of parts of claim 1, further comprising a pressure gauge for measuring the pressure in the enclosure.

14. (canceled)

15. The kit of parts of claim 1, wherein the first inflation/vacuum device is in communication with the enclosure on the first side portion, and the kit of parts further comprises a second inflation/vacuum device in communication with the enclosure on the second side portion.

16. (canceled)

17. A device for perihepatic packing, comprising:
a sac having a front wall, a back wall, and a periphery, wherein the front wall and back wall are joined together along the periphery to define an enclosure between the front wall and the back wall, wherein the periphery has a top edge and a bottom edge and the sac is foldable to a folded configuration in which the top edge and the bottom edge are brought towards each other, wherein the top edge comprises a notch extending inwardly towards the bottom edge and the sac has a first side portion on a first side of the notch and a second side portion on a second side of the notch, and wherein at least a portion of the sac is translucent;
at least one inflation/vacuum port in the sac for adding a fluid to the enclosure and/or for vacuuming a fluid from the enclosure; and
a first set of securing devices and a second set of securing devices along the periphery, for facilitating securing of the sac in the folded configuration.

18. The device of claim 17, wherein an entirety of the sac is translucent.

19. The device of claim 17, wherein the sac has a sac height between the top edge and the bottom edge, and the notch has a notch height, and the notch height is between one third and two thirds of the sac height.

20. The device of claim 17, wherein the first side portion is larger than the second side portion.

21. The device of claim 17, wherein the device comprises a first set of eyelets forming the first set of securing devices and a second set of eyelets forming the second set of securing devices.

22. (canceled)

23. (canceled)

24. The device of claim 17, wherein the at least one inflation/vacuum port comprises a first inflation/vacuum port on the first side portion, and a second inflation/vacuum port on the second side portion.

25. A method for perihepatic packing, comprising:
a. positioning a sac proximate a bleeding liver, the sac having a first side portion and a second side portion, a first edge extending along the first side portion and second side portion and defining a notch between the first side portion and second side portion, and a second edge extending along the first side portion and second side portion opposite the first edge;
b. tucking a section of the first side portion behind a right lobe of the liver;
c. tucking a section of the second side portion behind the left lobe of the liver;
d. positioning the notch to receive the hepatic veins;
e. bringing the section of the first side portion and the section of the second side portion around the liver towards the second edge, to a folded configuration;
f. securing the sac in the folded configuration; and
g. inflating the sac or vacuuming the sac to apply pressure to the liver.

26. The method of claim 25, wherein at least a portion of the sac is translucent, and the method further comprises viewing the liver through the sac to monitor the status of the liver.

27. The method of claim 26, further comprising adjusting an inflation level in the sac in response to the status.

28. The method of claim 25, wherein, step b. comprises tucking the section of the first side portion over the top of the liver and step c. comprises tucking the section of the second side portion over the top of the liver.

29. (canceled)
